(19) United States
(12) Patent Application Publication
(10) Pub. No.: US 2021/0093801 A1
(43) Pub. Date: Apr. 1, 2021

(54) DERMAL INJECTION GUIDE DEVICE

(71) Applicant: Neopellis LLC, Chicago, IL (US)

(72) Inventors: Tummuru Murali Krishna REDDY, Telangana (IN); Rajin AHUJA, Chicago, IL (US); Te-Shao HSU, Chicago, IL (US)

(21) Appl. No.: 17/033,921

(22) Filed: Sep. 28, 2020

Related U.S. Application Data
(60) Provisional application No. 62/906,780, filed on Sep. 27, 2019.

(51) Int. Cl. A61M 5/34; A61M 5/315; A61M 5/31; A61M 39/02

(52) U.S. Cl. CPC A61M 5/34; A61M 39/0208; A61M 5/3137; A61M 5/315

(57) ABSTRACT

A dermal injection guide including a needle housing unit comprising a guide tube including a semi-circular blade, a gripper, and a guide tube extension, which when combined with a syringe and cannula configures the dermal injection guide device. In its assembled state, the syringe and cannula is placed and secured in the needle housing unit. In operation, an incision point is made using the blade. Once the incision is made, the operator grips both the gripper and buttons located on the guide tube extension and applies force towards the distal end of the device. This, in turn, pushes the cannula through the needle housing unit and blade, and the cannula is inserted into the patient for the administration of dermal fillers into the dermal layer of the patient.

### CROSS-REFERENCE TO RELATED APPLICATIONS

This application claims priority to U.S. Provisional Patent Application Ser. No. 62/906,780, filed on Sep. 27, 2019, entitled “Dermal Injection Guide Device” currently pending, the entire disclosure of which is incorporated herein by reference.

### BACKGROUND

Dermal injection fillers are well-known in the art of dermatology and other medicinal practices and are often used to inject dermal fillers into a patient to increase volume or replace volume that may have been lost due to aging. Traditionally, these fillers have been injected using sharp tip needles. In sharp tip needle methods of injection, very thin, short, and sharp needles are used to inject dermal fillers or other active agents. When using these sharp tip needles, multiple injection points are often required, which increases the risk of hitting a vein or artery. In dermal injections, the goal of the procedure is not to inject the active agent into a vein or artery, but rather to inject the active agent into the dermal layer of the skin. To prevent accident exposure to arteries or veins, an alternative delivery method includes the use of blunt cannulas. In this method, blunt cannulas having a hollow bore which allows the fillers to pass through the tissue in the dermal layer and avoiding hitting a vein. Both the patient and the operator benefit from using the cannula method of injection as opposed to sharp tip needle methods.

Blunt end cannulas provide certain advantages over sharp tip needles for the injection of active agents in dermal injections. One such advantage of using blunt end cannula is in its length and flexibility. Blunt end cannulas are longer and more flexible than sharp tip needles, allowing the cannulas to cover a larger target area. With this larger target area, less injection points are required to achieve the desired results. Additionally, the blunt end cannulas may glide more softly and easily through the sub-cutaneous layer as compared to the sharp tip needles. Sharp tip needles are also prone to cutting through tissue and blood vessels, increasing the risk for bruising or blood vessel damage. The blunt end cannulas avoid this issue through its increased length and maneuverability, as well as its blunted tip. As such, filler injections with blunt end cannulas may be performed quickly, safely, and with less pain to the patient than sharp tip needles.

Despite the advantages the blunt end cannulas provide, this method does come with one major disadvantage. Blunt end cannulas are unable to pierce the skin of the patient due to the hollow and blunted tip. Current attempts to solve this issue are not without their own flaws. In one known cannula and needle assembly, the tip of the needle is placed in a forward mount which pierces the skin, and then the cannula is pushed through the skin. This method requires constant use by both hands of the operators and relies on thin strips of plastic and exposing the point of the cannula. This method exposes prolonged risk to the blunt cannula, through possible bending and breaking as the tip of the cannula is held static and force must be applied to the backward portion. When retracted, the blunt end of the cannula is entirely exposed, posing potential risks of inadvertent piercings.

Accordingly, a need exists for a device that utilizes the advantages and dermal injection properties of the blunt end cannula but that can also pierce the skin and safely administer an active agent to the dermal layer.

### SUMMARY OF THE INVENTION

The present invention is directed toward a device and method of safely and effectively administering a dermal filler or other active agent that generally includes a dermal injection device for the housing of a syringe to administer the reactive agent into a patient. The dermal injection device comprises a multi-component design, with individual components that, when assembled together, create the dermal injection guide. One embodiment of the dermal injection guide generally comprises four primary components: (1) a guide tube or hub to which components of the device connect, and which further serves as a housing unit for the syringe and can include a housing hub for a blade, such as a semi-circular blade; (2) a movable gripper initially located at or near a distal or administering end of the device and which may comprise flexible flaps for covering and protecting a sharp blade; (3) a guide tube extension located at or near a proximal or rearward end of the device that may (a) provide additional length to the device to accommodate the syringe, (b) include winged tabs or protruding tabs to aid a user in gripping the device, and (c) serve as a locking component that may prevent movement of the other components during use; and (4) a syringe and cannula for administering dermal fillers or other active agents.

In one embodiment, the dermal injection guide can be used to inject dermal fillers into the subcutaneous layer of a patient. Dermal injectors may be applied using a blunt cannula, that extends into the subcutaneous layer, bypassing arteries and veins and injects the fillers into the sub-cutaneous layer. Using a blunt cannula is particularly advantageous, as the risk to piercing, nicking, or otherwise cutting veins or arteries is greatly reduced. The advantage of using a blunt cannula in preventing nicking or cutting of arteries inversely provides a disadvantage in the initial piercing of skin. To combat the blunt cannula's disadvantages, while retaining its advantages, the dermal injection guide device of the present invention utilizes a separate blade to provide the initial piercing of skin, thereby providing an entry point for the cannula to safely enter into the subcutaneous layer of the skin.

When assembled in one configuration and position, the gripper provides cover and prevents direct exposure of the blade located at the distal or forward end of the needle hub. Just prior to the administration of dermal fillers, the gripper may be pushed from an initial or first, forward position to a retracted or second, rearward position by applying a rearward pressure. The gripper may then slide across the guide tube, guided by guide tube flap slots until a locking click or tactile sound is produced. This tactile sound confirms that the gripper is locked in placed and the device is ready to receive the syringe and cannula.

The syringe and cannula may then be inserted into a hollow opening defined in the proximal or rearward end of the guide tube until the syringe is locked in place. Prongs located in the interior of the gripper hub may create an interference fit with the syringe, thereby selectively locking and securing the syringe in place. In this initial position, the cannula is covered by the guide tube, thereby, protecting the cannula from exposure and potential damage.

Once the syringe and cannula have been placed and secured in the needle housing unit, the dermal injection guide is ready to begin the process of administering dermal fillers or other active reagents. When the gripper has been pulled back to its retracted or second position, and located generally at the proximal end of the dermal injection guide, this exposes the blade located at the forward or distal end of the device. The operator may guide the device into position and, using with the exposed blade, pierce the skin of the patient. Once the skin has been pierced, the operator may then press down on the buttons located on the needle hub extension, thereby releasing the tactile tabs and locks from the gripper tactile and lock tabs. Once released, the operator may apply a forward pressure to the gripper, moving the gripper back towards its original position. Because the syringe is secured to the gripper, this in turn moves the syringe and cannula towards the forward or distal end of the device. As the gripper, cannula, and syringe move closer to the distal end, the cannula begins to become exposed as the terminal end of the cannula exits from the guide tube. This extension continues until the gripper is returned to its original position and the gripper is locked back in place. Once fully extended, the cannula can be inserted into the opening created by the blade and can be inserted into the dermal layer of the patient. When the desired location of the end of the cannula is reached, the operator may then press on the plunger located on the syringe to administer dermal filler or other active agent into the patient.

After application, the cannula may be removed from the patient, and in a manner following the steps previously performed, the operator may grab onto the gripper and pull the gripper, syringe, and cannula back to the proximal end of the device, thereby protecting the cannula from damage. This process may be repeated once or a plurality of times, depending on how much filler is desired and the locations on the patient that require injection.

### BRIEF DESCRIPTION OF THE SEVERAL VIEWS OF THE DRAWING

In the accompanying drawings, which form a part of the specification and are to be read in conjunction therewith in which like reference numerals are used to indicate like or similar parts in the various views:

FIG. 1 is a perspective side view of a needle housing unit illustrating the position of the components with a gripper in a first or forward position at a distal end in accordance with one embodiment of the present invention;

FIG. 2 is a perspective side view of a needle housing unit of FIG. 1 illustrating the position of the components after the gripper is moved to a second or retracted position toward the proximal end;

FIG. 3 is a perspective side view of a needle housing unit of FIG. 2 and a syringe and cannula in a partially pre-assembled orientation in accordance with one embodiment of the present invention;

FIG. 4 is a perspective side view of the needle housing unit and a syringe and cannula in a first assembled orientation;

FIG. 5A is a cross-sectional side view of the needle housing unit, syringe, and cannula of FIG. 4;

FIG. 5B is an enlarged cross-sectional side view of the needle housing unit of FIG. 5A demonstrating the positioning of the cannula relative to the blade;

FIG. 6 is a side view of the needle housing unit, syringe, and cannula of FIG. 4 illustrating the gripper and cannula in an extended position;

FIG. 7A is a cross-sectional side view of the needle housing unit, syringe, and cannula of FIG. 6;

FIG. 7B is an enlarged cross-sectional side view of the needle housing unit of FIG. 7A demonstrating the positioning of the cannula relative to the blade; and

FIG. 8 is an exploded view of the needle housing unit, syringe, and cannula in accordance with one embodiment of the present invention.

### DESCRIPTION OF THE INVENTION

The invention will now be described with reference to the drawing figures, in which like reference numerals refer to like parts throughout. For purposes of clarity in illustrating the characteristics of the present invention, proportional relationships of the elements have not necessarily been maintained in the drawing figures. It will be appreciated that any dimensions included in the drawing figures are simply provided as examples and dimensions other than those provided therein are also within the scope of the invention.

The following detailed description of the invention references specific embodiments in which the invention can be practiced. The embodiments are intended to describe aspects of the invention in sufficient detail to enable those skilled in the art to practice the invention. Other embodiments can be utilized, and changes can be made without departing from the scope of the present invention. The disclosure also includes all such components, features, parts, and operations referred to or indicated in this specification, individually or collectively, and any and all combinations of any one or more of such steps or features. One or more features shown and discussed with respect to one embodiment may be included in another embodiment even if not explicitly described or shown with another embodiment. The terminology used herein is for the purpose of describing particular embodiments only and is not intended to be limiting of the invention. The present invention is defined by the appended claims and the description is, therefore, not to be taken in a limiting sense and shall not limit the scope of equivalents to which such claims are entitled.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references 70 to a structure or feature that is disposed “adjacent” another feature may have portions that overlap or underlie the adjacent feature.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The present invention generally relates to an injection device or dermal injection guide device 10 for the administration of active agents. Active agents as used herein, can refer to dermal fillers, vaccines, or other medicinal or pharmaceutical liquids or gases.

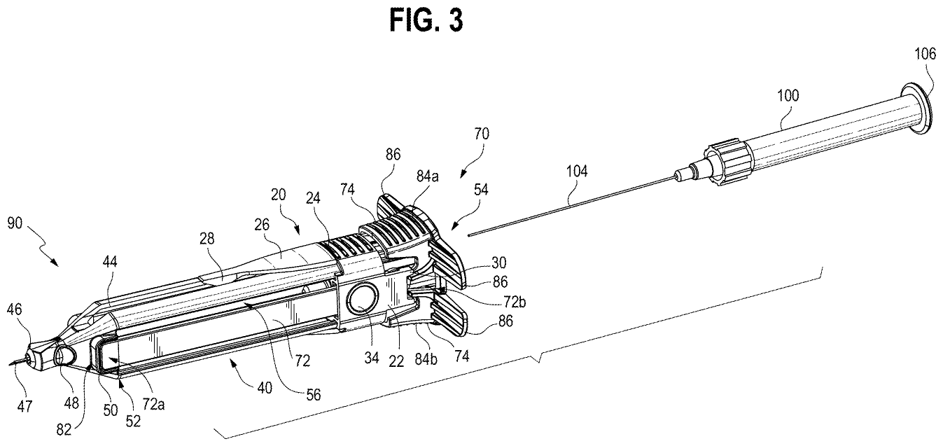

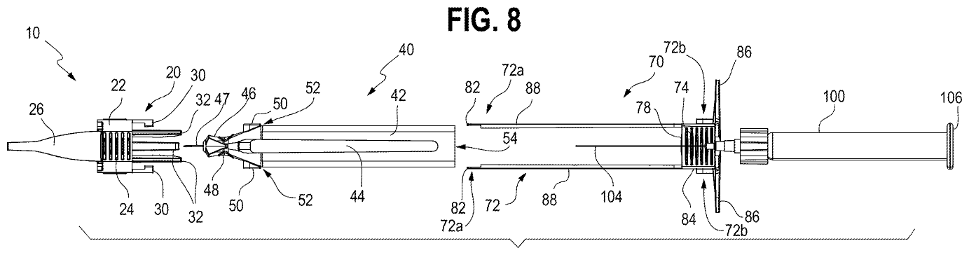

As generally depicted in FIGS. 3-8, dermal injection guide device 10 is comprised of a guide tube 40, a gripper 20, a guide tube extension 70, a needle housing unit 90, and a syringe 100 with a removably attached cannula 104. Needle housing unit 90 may be combined with syringe 100 and cannula 104 to create dermal injection guide device 10, which can be used to inject active agents into a patient. The general alignment of needle housing unit 90, syringe 100, and cannula 104 is depicted in the exploded view of FIG. 8. It will be appreciated that in one preferred embodiment of the present invention the primary subject will be a human patient, but the present invention may also be used with animals or a variety of other subjects. It will be further appreciated that dermal injection guide device 10 may be used in the administration of a variety of different active agents, which may require administration in differing parts and points in the body and which may further require differing apparatus for application.

The illustrated in embodiment depicts a dermal injection guide device 10 for the administration of dermal fillers into the sub-cutaneous or dermal layer, through the use of a cannula 104. It will be appreciated that the cannula may be any suitable type of cannula or similar structure, including but not limited to, a blunt end cannula, a blunt tip cannula, a smooth tip cannula, a microcannula, or the like, including those cannulas currently known or hereafter developed. The cannula 104 may be a any suitable length and diameter. However, it will also be appreciated that dermal injection guide device 10 may be adapted for and be used with other medical syringes and apparatuses and can be further compatible with other active agent delivery systems other than through a cannula 104. As used herein, cannula 104 can be interchangeably used with other delivery apparatuses now known in the art or have yet to be discovered or invented. By way of non-limiting example, dermal injection guide device 10 may be used with a hypodermic needle for delivery into arteries or veins. Even further, dermal injection guide device 10 can further be used to deliver vaccines, or other medications, in addition to dermal fillers. It will be appreciated that dermal injection guide device 10 could even be particularly beneficial in the delivery of a vaccine or treatment for SARS-CoV-2 (also known as COVID-19 or novel coronavirus).

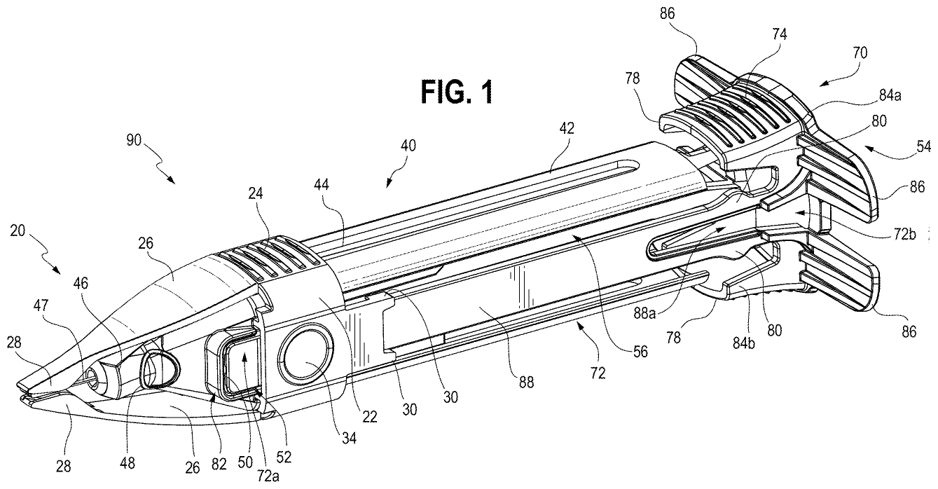

As demonstrated in FIGS. 1-3, needle housing unit 90 may generally comprise a guide tube 40, guide tube extension 70, blade housing 46, blade 47, and gripper 20. Guide tube 40 may act as a central hub or attachment point for the remaining components of dermal injection guide device 10. In addition to acting as a central hub or framework, guide tube 40 can further be used to guide syringe 100 and cannula 104 to specific locations of a patient to deliver the active agent dose. In the illustrated embodiment, guide tube 40 may generally comprise a rectangular geometrically shaped guide tube structural body 42, but it will be appreciated that the geometric design and shape of guide tube structural body 42 may vary depending on the embodiment. By way of nonlimiting examples, the cross-section of guide tube structural body 42 could be circular, oval, polygonal, triangular, pentagonal, hexagonal, or a variety of other suitable shapes and designs to accommodate a syringe 100 and cannula 104. As with the geometric shape, the overall length and width of guide tube 40 may vary based on the design. As the length, height, width, and shape, and overall size of medical syringes can greatly vary, dermal injection guide device 10 can be equally as versatile in its shape and size to accommodate a variety of medical syringes available. As such, it will be appreciated that guide tube 40 will generally comprise a corresponding length, height, and width to accommodate syringe 100 for which it is to be used with. Guide tube 40 may be made of plastic, polymer, aluminum, metal, or other suitable types of materials.

Guide tube 40 may comprise a hollow end 54 with an opening defined as its rearward or proximal end, and a pathway for receiving the syringe 100 therein. As described in greater detail herein, hollow end 54 allows syringe 100 and cannula 104 to be placed into and pass through guide tube 40 during operation. Guide tube 40 may comprise two opposing sides that are solid design and can further comprise two opposing sides incorporating gaps or longitudinally extending guide tube structural body opening 56 defined in guide tube 40. It will be appreciated that guide tube 40 may also be designed as a singular piece with no gaps between the top and bottom of guide tube 40.

In addition to acting as a central hub, guide tube 40 may further be used in providing the initial piercing of skin, which allows cannula 104 to enter into the dermal layer of the patient leading to the application of cannula 104 and ultimately the administration of the active agent. As demonstrated in FIGS. 1-8, guide tube 40 comprises a protruding or otherwise extending blade housing 46 located at the distal or forward end of guide tube 40. As used herein, “forward” and “distal” ends refer to the end of dermal injection guide device 10 what will face towards the patient. As further used herein, “rearward” and “proximal” ends refer to the end of the dermal injection guide device 10 that will face away from the patient. Blade housing 46 may be used to support, stabilize, hold, or otherwise house a blade 47, such as a semi-circular blade or semi-cylindrical blade, for example. As described in greater detail below, blade 47 may be used for the initial piercing of the dermal layer of skin of the patient. A blunt needle, such as blunt cannula 104, is generally incapable of piercing skin, and a sharp hypodermic needle can be prone to breaking or snapping during the piercing of skin. Blade 47, comprising a sharp blade and point is capable of piercing skin but is not prone to breaking or snapping. In one embodiment of dermal injection guide device 10, blade 47 comprises a generally semi-circular, semi-cylindrical or crescent design, becoming more narrow until reaching a sharpened tip. However, it will be appreciated that blade 47 could comprise a full circular or cylindrical design, similar to that of a hypodermic needle. One advantage of a semi-circular design, and described in greater detail herein, is that cannula 104 may rest against semi-circular blade 47 optionally creating a general interference fit. This interference fit may aid in both stabilizing cannula 104, as well as providing a guide path for cannula 104 as it is inserted in the patient. Blade 47 may optionally be permanently secured to blade housing 46 or it may be alternatively be removably attached to blade housing 46. Even further, blade 47 may be retractable, and may be housed entirely in blade housing 46 until ready for use and extend out. Depending on the embodiment, blade 47 may be secured through adhesive methods, mechanical methods, molded directly into blade housing 46, or secured by any other suitable method. Blade housing 46 may further comprise at least one guide tube grip 48, located on one or multiple sides of blade housing 46. Depending on the embodiment, guide tube grip 48 may comprise a generally embossed circular design to provide an additional point of contact for the operator to manipulate or otherwise handle dermal injection guide device 10. It will be appreciated that guide tube grip 48 could comprise differing shapes and geometric designs and may include grooves defined in blade housing 46 or may alternatively comprise a textured surface.

In an embodiment of guide tube 40 comprising a generally rectangular design, one or more side of guide tube structural body 42 may comprise a generally hollow side or side otherwise defined by a guide tube structural body opening 56. However, in other embodiments, guide tube structural body 42 may be constructed with entirely solid sidewalls without openings defined therein. Guide tube 40 may further comprise at least one longitudinally extending guide tube flap slot 44, located on guide tube structural body 42, acting as a channel to help guide gripper 20 during operation. As demonstrated in FIGS. 1-4 and 6, guide tube flap slot 44 may be located on a generally solid portion of guide tube structural body 42, adjacent to a side comprising a guide tube structural body opening 56. Further, in an embodiment comprising multiple guide tube flap slots 44, guide tube flap slots 44 may be located on opposing sides of guide tube 40, or may be located on adjacent sides of guide tube 40. As further described below, guide tube flap slots 44 may be configured to accommodate gripper 20, or a portion thereof, for final configurations and assembly. Additionally, in one embodiment of the present invention, guide tube 40 can further comprise grips, ridges, tactile gripping textures, or guide tube serrations 50 on at least one side of guide tube 40. Such guide tube serrations 50 may aid the operator in use of the dermal injection guide device 10 by providing a gripping texture to avoid slippage. As demonstrated in FIGS. 1-4, 6, and 8, guide tube serrations 50 may be located on the forward or distal end of guide tube 40 comprising blade housing 46. Guide tube serrations 50 can be located on at least one side of guide tube 40, but guide tube serrations 50 may alternatively be located on two sides, three sides, or more sides of guide tube 40. Additionally, guide tube 40 may further comprise at least one defined groove 52 for holding guide tube extension 70 in place in the final assembly and configurations. As described further herein, defined groove 52 can be adapted for accepting guide tube extension lips 82. It will be appreciated that number and location of defined grooves 52 may vary based on the embodiment, and it will further be appreciated that defined grooves 52 may be of a different design, and may vary based on the embodiment of the present invention, and may be circular, triangular, or other shapes. In the illustrated embodiment, guide tube 40 may comprise two defined grooves 52 located on opposing sides of guide tube structural body 42, and may be generally located near the forward end of guide tube 40. Even further, defined grooves 52 may be located generally on the interior and generally in the same positioning to guide tube serrations 50. However, it will be appreciated that defined grooves 52 may be located in multiple points and locations in guide tube 40 depending on the embodiment.

As designed, guide tube 40 can provide the operator with ergonomically designed gripping and handling support. It will further be appreciated that one embodiment of guide tube 40 provides structural support and optimal positioning of gripper 20, the guide tube extension 70, syringe 100 and cannula 104, and blade 47.

In one embodiment of the present invention, dermal injection guide device 10 may further comprise a gripper 20. Gripper 20 may be used to grab and handle syringe 100 and cannula 104 safely and effectively, as well as to cover blade 47 prior to the procedure and cannula 104 at the end of the procedure. Gripper 20 may generally comprise a hollow ring design, conical design, or gripper hub 22 design. Gripper hub 22 may alternatively be designed to have a circular, oval, polygonal, triangular, pentagonal, hexagonal, or a variety of other suitable shapes and designs so that it may be generally compatible with the geometric shape of guide tube 40. In other words, gripper hub 22 will generally comprise the same geometric shape, size, and design of guide tube 40 and syringe 100. Gripper hub 22 may also be used to accommodate a syringe 100 and cannula 104 with an area to actuate dermal injection guide device 10, and further guide cannula 104. Gripper 20 may even further comprise at least one side comprising a gripping texture, and as demonstrated in the figures, the gripping texture is located on the exterior of gripper hub 22. In one embodiment of the present invention, the gripping texture may be comprised of a plurality of gripper serrations 24. It will be appreciated that the gripping texture may also be designed in a cross-pattern, a plurality of bumps, or further patterns, designs, and material that will aid the user in gripping dermal injection guide device 10. Gripper 20 may optionally and additionally include an additional contact point on at least one other side from gripper serrations 24 for added support and maneuverability. In such an embodiment, this additional contact point may comprise an embossed shape, such as an embossed ring 34, or it may include additional gripping texture. Gripper 20 may be made of a plastic, polymer, aluminum, metal, or other type of suitable material.

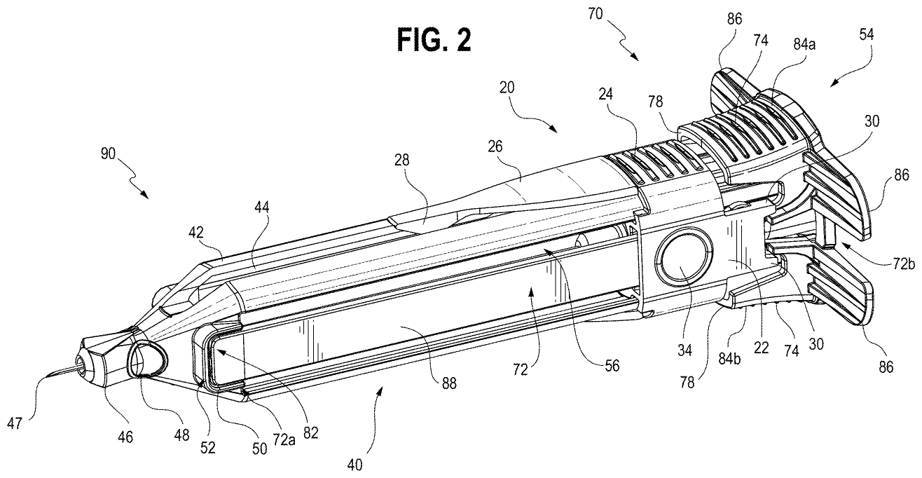

As demonstrated in FIGS. 1-4, 5A, 6, 7A, and 8, one embodiment of gripper 20 may further comprise at least one external gripper flap 26 for protecting and preventing exposure of blade 47 housed in guide tube 40 before, during, and after injection. In the illustrated embodiment, gripper 20 may comprise two gripper flaps 26 located at opposing sides of gripper 20. However, it will be appreciated that the actual number of gripper flaps 26 may be utilized depending on the embodiment of dermal injection guide device 10 and may be arranged in multiple positions around gripper hub 22. Gripper flaps 26 may be made of plastic or other suitable type of material, however gripper flaps 26 should be constructed from a generally flexible material allowing gripper flaps 26, to flex and conform to the shape of guide tube structural body 42 when the grippe 20 is pulled back from an initial position, for example as shown in FIG. 2. When gripper 20 is repositioned to its proximal location, gripper flaps 26 take on a generally flat profile, which is aided by the flexibility of gripper flaps 26. This flexibility aids in sliding or otherwise repositioning gripper 20 along guide tube structural body 42. Because the overall size of dermal injection guide device 10 may vary based on embodiment, and on the corresponding size of syringe 100, the length and width of gripper flaps 26 may be of any suitable dimension and can vary from embodiment to embodiment. Further, gripper flaps 26 may also be designed in a variety of shapes and designs and, in the illustrated embodiment, gripper flaps 26 may be designed in a generally curved, domed shape. However, it will be appreciated that other geometric shapes and designs are possible for gripper flaps 26. In certain embodiments, such as the illustrated embodiment, gripper flaps 26 may further comprise at least one gripper flap shrouds 28 to further protect blade 47. Gripper flap shrouds 28 may generally extend from the outer perimeter from one or more of gripper flaps 26. Gripper flap shrouds 28 may vary in design, but they may be configured based on the design of gripper flaps 26 and provide additional side protection to blade 47. Gripper flap shrouds 28 may further be used to aid in adjusting the position of gripper 20 during operation. Gripper flap shrouds 28 may align with and be inserted into guide tube flap slots 44 located on guide tube structural body 42. By aligning gripper flap shrouds 28 with guide tube flap slots 44, gripper 20 may maintain a constant path as it is moved from its initial position at the forward or distal end of needle housing unit 90 and to its secondary position located at the rearward or proximal end of needle housing unit 90.

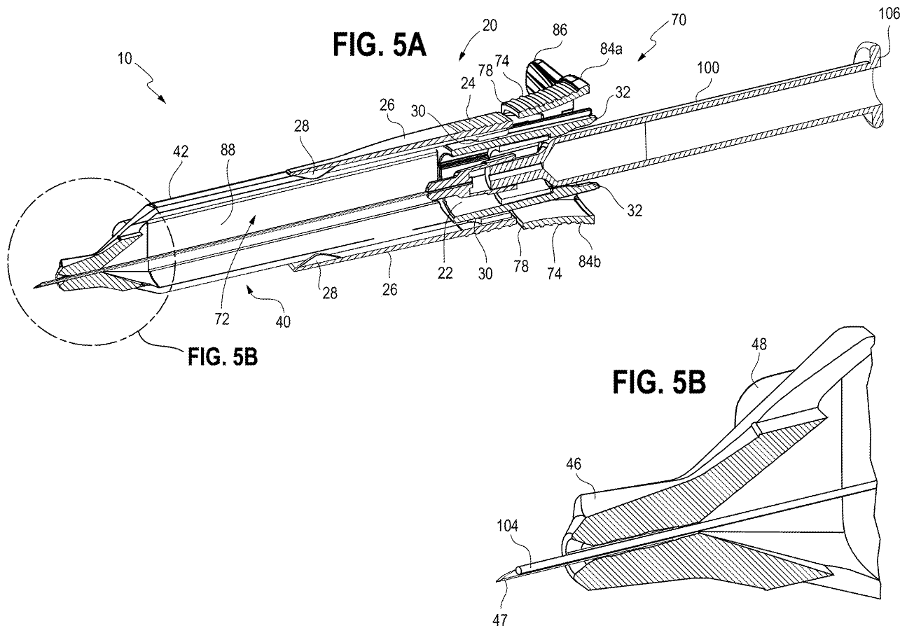

Additionally, gripper 20 may further comprise one or more gripper tactile and lock tabs 30 located in the interior of gripper hub 22. Gripper tactile and lock tabs 30 may be rectangular tabs shaped for guiding syringe 100 and cannula 104 and for locking gripper 20 in place at the proximal or rearward end of needle housing unit 90, however, gripper tactile and lock tabs 30 may comprise other geometric shapes and designs. In one embodiment, gripper hub 22 may comprise four gripper tactile and lock tabs 30, however, it will be appreciated that any suitable number of gripper tactile and lock tabs 30 may be utilized. As best illustrated in FIGS. 5A, 7A, and 8, gripper 20 may additionally comprise one or more gripper prongs 32 located in the interior of gripper hub 22 and which may extend inward towards the proximal end of needle housing unit 90. Gripper prongs 32 may generally comprise a shallow hook design or other design, capable of creating an interference grip, locking connection, securement, or other engagement with syringe 100. In one embodiment, gripper 20 may comprise six gripper prongs 32, however, it will be appreciated that any suitable number of gripper prongs 32 may be utilized. Gripper prongs 32 may generally be placed circumferentially around the interior of gripper hub 22 and in a manner to both surround and secure syringe 100 when placed in the interior of needle housing unit 90. Further, gripper prongs 32 should further be placed in a manner such that an interference or locking fit is created between gripper hub 22 and syringe 100. This interference fit secures syringe 100 to gripper hub 22 and, therefore, gripper 20 will aid in use of dermal injection guide device 10 and ultimate application of active agent into the patient.

Dermal injection guide device 10 may also comprise a guide tube extension 70. As generally demonstrated in FIGS. 1-4, 5A, 6, 7A, and 8, guide tube extension 70 comprises a generally asymmetric geometric design, comprising an inserting end 72a, a terminal end 72b and a structural body 72 connecting the inserting end 72a and terminal end 72b. In one embodiment, guide tube extension 70 comprises a generally forked or split design, thereby, consisting of two guide tube extension limbs 88 of guide tube extension structural body 72. At inserting end 72a there may be at least one guide tube extension lip 82 corresponding to one or both guide tube extension limbs 88 of guide tube extension structural body 72. The shape of guide tube extension lips 82 may vary based on the embodiment of the invention and may be rectangular, circular, pentagonal, or other shapes. However, it will be appreciated that the design of guide tube extension lips 82 will generally correspond to and be compatible with defined grooves 52 in guide tube 40, such that guide tube extension lips 82 can be placed in and secured to defined grooves 52, thereby, locking guide tube extension 70 in place. In the illustrated embodiment, guide tube extension 70 may be removably inserted into and secured in the interior of guide tube 40, however, it will be appreciated that in alternative embodiments guide tube extension 70 could be removably connected and secured to the exterior of guide tube 40. By placing and securing guide tube extension lips 82 in defined grooves 52, it will be appreciated that guide tube extension can be locked in place for increased stability and security during operation of dermal injection guide device 10. It will be further appreciated that, depending on the embodiment, guide tube extension 70 could be permanently attached and secured to guide tube 40. In one embodiment of the present invention, as demonstrated in FIGS. 1-4, 5A, 6, 7A, and 8, guide tube extension limbs 88 could align generally with opening 56 of guide tube 40, thereby defining a perimeter sidewall of needle housing unit 90. However, it will be appreciated that in an embodiment without opening 56 defined in guide tube 40, guide tube extension limbs 88 could be partially or complexly enclosed by guide tube structural body 42. Guide tube extension 70 may be made of plastic, aluminum, metal, or other suitable types of materials.

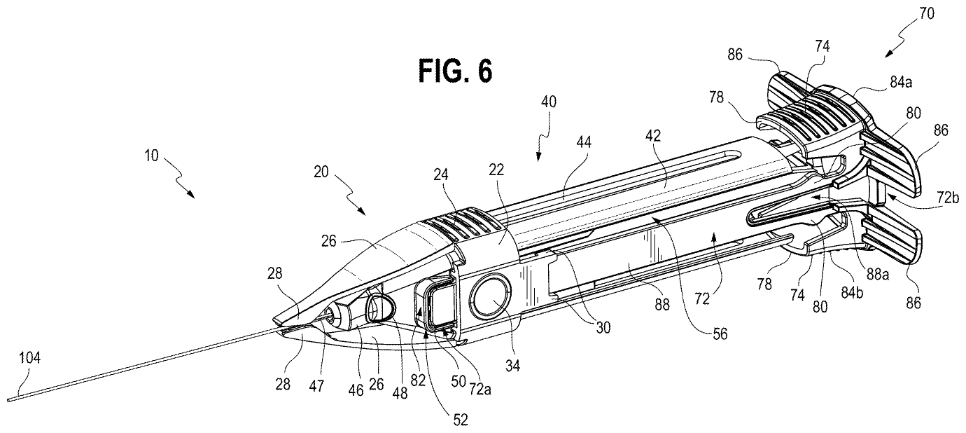

Along each side of guide tube extension structural body 72, or guide tube extension limbs 88 in an embodiment comprising such, there may be at least one opening, a guide tube extension middle groove 88a. In one embodiment of the present invention, guide tube extension structural body 72 comprises one guide tube extension middle groove 88a on each of the two guide tube extension limbs 88, located near the proximal end of guide tube extension but before guide tube extension buttons 84. The guide tube extension middle grooves 88a may provide flexibility and a spring action for locking and unlocking the present invention. As illustrated in FIGS. 1, and 6, near the proximal or rearward end of guide tube extension structural body 72, but before guide tube extension buttons 84, there may be at least one guide tube tactile tabs and lock 80. In one embodiment of the present invention, there can be four guide tube tactile tabs and locks 80, with one tactile tab and lock 80 located at the top of both forks of the two guide tube extension limbs 88 and one tactile tab and lock 80 located at the bottom of both forks of the two guide tube extension limbs 88.

The proximal end of guide tube extension structural body 72 may further comprise a blocking segment, of a wider width than guide tube extension structural body 72. This proximal segment may function as guide tube extension buttons 84 and may be used to release syringe 100 from its locked position and allowing syringe 100 to be removed from dermal injection guide device 10. Guide tube extension buttons 84 may further comprise a textured surface or guide tube extension serrations 74 for added support and textured grip for the operator. Guide tube extension serrations 74 may be located on each of guide tube buttons 84 or may be located on only one of guide tube buttons 84, on either the upper 84a or lower guide tube button 84b.

Guide tube extension buttons 84 may optionally, and additionally, comprise at least one extending wing or flange 86 for aiding the user in operation. As depicted in FIGS. 1-4, 5A, 6, 7A, and 8, flange 86 may extend perpendicularly away from guide tube extension button 84. As further depicted, each guide tube extension button 84 may comprise two flanges 86 on opposing sides of guide tube extension button 84.

Guide tube buttons 84 may further comprise at least one guide tube extension stopper 78, located on the side of guide tube buttons 84 facing towards guide tube lips 82. In one embodiment of the present invention, there may be two guide tube extension stoppers 78, one located on the top guide tube button 84a and one located on the bottom guide tube button 84b. Guide tube extension stoppers 78 may be used to reduce the friction between gripper 20 and syringe 100 when in the extended position.

In one embodiment of the present invention, needle housing unit 90 may be assembled in the following manner. Guide tube 40 may be the central component of needle housing unit 90. Gripper 20 may be placed and secured near the front of guide tube 40, near blade housing 46, such that gripper flaps 26 and gripper flap shrouds 28 provide cover to protect blade 47. This initial positioning is illustrated in FIG. 1. The positioning of gripper 20 may provide a cover to protect blade 47 located in or extending from blade housing 46. Guide tube extension 70 may be located in the interior of guide tube 40, and may be locked in place by inserting guide tube extension 70 inside the hollow interior of guide tube 40 and then by locking guide tube extension lips 82 into defined grooves 52. When needle housing unit 90 is combined with syringe 100 and cannula 104, it will be appreciated that this combined device forms dermal injection guide device 10.

In one embodiment of the present invention, dermal injection guide device 10 may be used in a method of injecting dermal filler or other active agents to the dermal layer of a patient, in a manner demonstrated. In operation, operator may grip dermal injection device 10, by gripper serrations 24 or embossed grip 34 with one hand and guide tube extension buttons 84 with the other. At this time, needle housing unit 90 is in its original configuration as illustrated in FIG. 1. As described herein, guide tube extension flanges 86 may aid the operator in gripping guide tube extension buttons 84. By pressing down on gripper serrations 24 and guide tube extension buttons 84, the operator may apply a rearward pressure and then pull gripper 20 towards the proximal end of needle housing unit 90. The operator will continue to pull gripper 20 towards the proximal end of needle housing unit 90 until gripper 20 is locked in place and an audible click or other sound is made. Gripper 20 may lock in place when gripper lock and tabs 30 reach and connect with guide tube extension tabs and lock 80. When the haptic click, audible click, or other sound is made, this alerts the operator that needle housing unit 90 is in its secondary configuration as illustrated in FIG. 2. Alternatively, the operator after pressing down on gripper serrations 24 and guide tube extension buttons 84 could instead apply forward pressure, and push guide tube 40 and guide tube extension 70 forward, toward the distal end of needle housing unit 90. However, it will be appreciated that no matter which method is used, the positions of gripper 20, guide tube 40, and guide tube extension 70 will be the same.

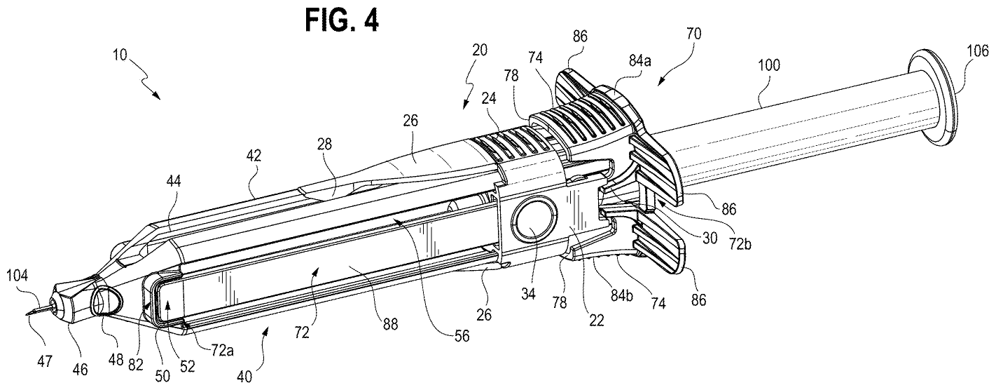

The operator may then take a combined syringe 100 and cannula 104 and align with hollow end 54 of needle housing unit 90. This is generally depicted in FIG. 3. After alignment, the operator may then insert the combined syringe 100 and cannula 104 into needle housing unit 90, thereby, providing final configuration and assembly of dermal injection guide device 10. This configuration is depicted in FIG. 4. Syringe 100 and cannula 104 may be guided through the interior of needle housing unit 90 until syringe 100 reaches gripper hub 22. When syringe 100 reaches gripper hub 22, gripper prongs 32 may connect with syringe 100, creating an interference or locking fit, and thereby, locking or otherwise securing syringe 100 in place. With syringe 100 locked in place, cannula 104 may rest inside blade 46 housing and, in one embodiment of the present invention, cannula 104 may be completely encased inside blade housing 46 with no portion of cannula 104 protruding out. In another embodiment of the present invention, cannula 104 may protrude out from blade housing 46 and may rest inside the crescent shape of semi-circular blade 47. Further in this embodiment, cannula 104 will not extend past the terminal edge of blade 47. This feature is illustrated in the cross-sectional of FIGS. 5A and 5B. In an even further embodiment, cannula 104 may rest inside the crescent shape of blade 47 but extend further past the terminal edge of blade 47.

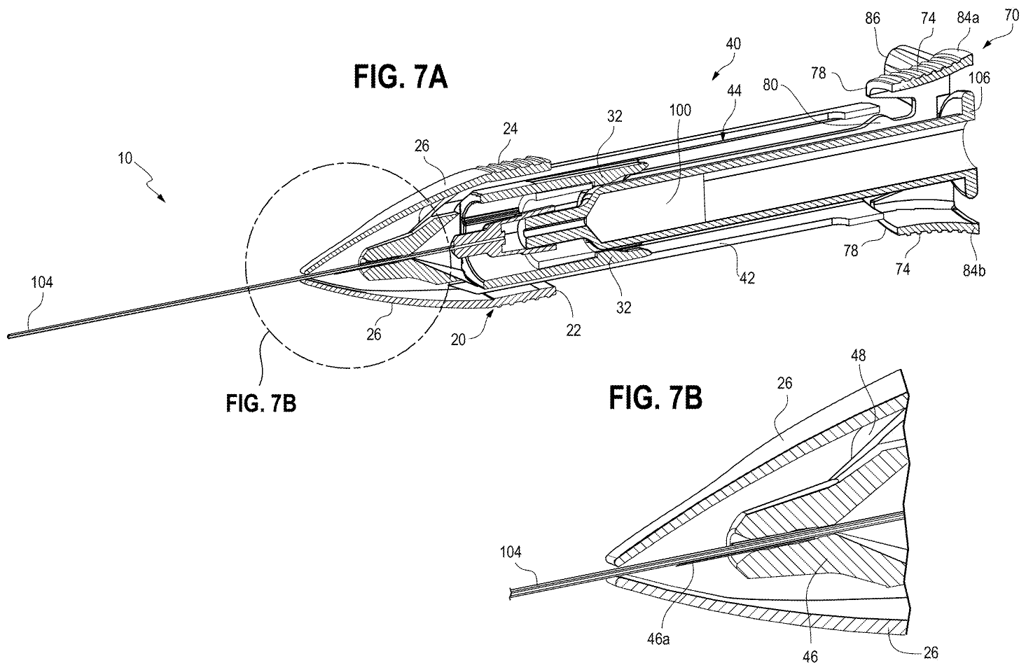

With syringe 100 and cannula 104 placed and secured in needle housing unit 90, the operator may then hold the dermal injection guide device 10 by gripping on gripper serrations 24 and/or embossed grip 34 for tactile support with one or more hands. The operator may then pierce the skin of the patient using blade 47. After piercing the skin, the operator may then retract guide tube 40 covering cannula 104 by pressing on guide tube extension buttons 84. This may release guide tube tactile tabs and lock 80 from gripper lock and tabs 30. When released, gripper 20 and attached syringe 100 and cannula 104 may be pushed forwards towards the distal end of the dermal injection guide device 10, where blade 47 is located. As gripper 20 is pushed back to its original location, syringe 100 and cannula 104 is pushed along with it. This may cause syringe 100 to be pushed closer and closer to blade 47 and push the terminal edge of cannula 104 past the terminal edge of blade 47 and closer to the final application site. This is continued until gripper 20 is returned to its original positioning and the maximum amount of cannula 104 has been exposed. This is configuration is demonstrated in FIGS. 6, 7A, and 7B. Cannula 104 may be guided by blade 47 and may continuously or sporadically touch the interior surface of blade 47. It will be appreciated that, because syringe 100 and cannula 104 are held in place by gripper hub 22, no portion of syringe 100 itself will protrude from outside guide tube 40. Only cannula 104 will protrude out from guide tube 40. Cannula 104 may then be inserted into the dermal layer of the patient, through the same hole created by blade 47. Once cannula 104 has been inserted into the desired position in the dermal layer, the operator may then press on a plunger rod 106 of syringe 100 injecting the dermal filler or other active agent. After the desired amount of filler has been injected, the operator may grip the dermal injection guide device 10 and pull it and cannula 104 out from the patient.

It will be appreciated that the above-described steps may be performed once or a plurality of times, depending on how much filler is desired and the locations on the patient required. After injection has been completed, the operator may then grab the dermal injection guide device 10 by holding onto gripper serrations 24 and pressing down on guide tube extension button 84 and pulling gripper 20 towards the proximal end. It will be appreciated that this will, in turn, pull cannula 104 and syringe 100 towards the proximal end as well, covering cannula 104 with guide tube 40. By covering cannula 104 with guide tube 40 after use, cannula 104 is protected from damage and cannula 104 is covered preventing accidental piercings. It will be appreciated that cannula 104 and syringe 100 may then be removed from dermal injection guide device 10 and replaced with a new cannula 104 and syringe 100 for additional doses following the above mentioned method or the dermal injection guide device 10 may be placed in storage following the final dosage.

It will be further appreciated that the overall design of dermal injection guide device 10 is capable of use with a variety of medical and pharmaceutical delivery apparatus. By way of non-limiting example, cannula 104 could be replaced with a traditional hypodermic needle. In this alternative embodiment, an active agent can be injected into a patient's artery or vein or into the muscle rather than into the patient's dermal layer. It will be further appreciated that dermal injection guide device 10 may further be used with other active agents other than dermal fillers. By way of non-limiting example, dermal injection guide device 10 could be used in the administration of a vaccine, insulin, or other medicinal or pharmaceutical agents.

From the foregoing, it will be seen that this invention is one well adapted to attain all the ends and objects hereinabove set forth together with other advantages which are inherent to the structure and method. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is, therefore, contemplated that other modifications and applications, or equivalents thereof, will occur to those skilled in the art.

The articles “a,” “an” and “the” are used to refer to one or to more than one (i.e., to at least one) of the grammatical object of the article. As used herein, the singular forms “a,” “an” and “the” are intended to include the plural forms as well, unless the context clearly indicates otherwise. The terms “having” and “including,” and similar terms as used in the foregoing specification are used in the sense of “optional” or “may include” and not as “required.” Throughout this specification, unless the context requires otherwise the word “comprise,” and variations such as “comprises” and “comprising,” will be understood to imply the inclusion of stated features, integers, steps, operations elements, and/or components but do not preclude the presence or addition of one or more other features, integers, steps, operations, elements, components, and/or groups thereof. The terms “comprise” and “comprising” are used in the inclusive, open sense, meaning that additional elements may be included. It is not intended to be construed as “consists of only,”

Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. An injection device comprising:
a housing unit including a proximal end and a distal end, the housing unit comprising:
a guide tube defining a pathway for receiving a syringe therein; and
a blade adapted for protruding from a distal end of the housing unit.

2. The device of claim 1 further comprising the syringe that includes a cannula extending from a forward end of the syringe.

3. The device of claim 1 further comprising a gripper having a hub at least partially surrounding the guide tube and configured for being selectively movable between a first position proximate the distal end of the housing unit and a second position proximate the proximal end of the housing unit.

4. The device of claim 3, wherein the guide tube defines at least one longitudinally extending opening for receiving at least a portion of the gripper therein.

5. The device of claim 3, wherein the hub further comprises at least one flap extending from the hub for at least partially covering the blade when the gripper is in the first position.

6. The device of claim 5, wherein the at least one flap includes at least one shroud for additional protection of the blade when the gripper is in the first position and for being received by a longitudinally extending slot defined in the guide tube as the gripper is being moved toward the second position.

7. The device of claim 3, wherein the gripper has at least one tab for selectively locking the gripper in the second position.

8. The device of claim 3, wherein the gripper has at least one internal prong for engaging the syringe and securing the syringe to the hub.

9. The device of claim 8, wherein the gripper is adapted for moving the syringe from the proximal end of the housing unit to the distal end of the housing unit when the gripper is moved from its second position to its first position.

10. The device of claim 9, wherein the gripper is adapted for moving the syringe from the distal end of the housing unit back to the proximal end of the housing unit when the gripper is moved from its first position to its second position.

11. The device of claim 1, wherein the blade is a semi-circular blade configured for selectively receiving at least a portion of a cannula therethrough.

12. The device of claim 11, wherein the blade is configured to pierce skin of a subject to create an entry point for the cannula and the cannula is configured to be extended into the subject and to deliver an active agent in a subcutaneous or dermal layer.

13. The device of claim 1 further comprising a guide tube extension adapted to be inserted and locked into the guide tube.

14. The device of claim 13, wherein the guide tube extension includes lips adapted for being received within grooved defined in the guide tube.

15. The device of claim 13, wherein the guide tube extension includes defined grooves in order to achieve a flexible and spring action.

16. The device of claim 13, wherein the guide tube extension includes at least one lock for selectively locking a gripper in a second position.

17. A method of using an injection device comprising the steps of:
providing an injection device comprising:
a guide tube having a proximal end and a distal end, and defining a interior pathway for receiving a syringe therein;
a blade protruding from a distal end of the guide tube;
a gripper configured for being selectively movable between a first position proximate the distal end of the guide tube and a second position proximate the proximal end of the guide tube;
moving the gripper from the first position to the second position in which the gripper can be locked;
inserting a syringe assembly with a cannula into the interior pathway of the guide tube until the syringe assembly is secured to the gripper;
penetrating the skin of a subject with the blade;
unlocking the gripper and moving the gripper with syringe assembly secured thereto toward the distal end of the guide tube;
guiding the cannula into a dermal layer of the subject;
injecting active agent into the dermal layer by pushing a plunger of the syringe; and
pulling the gripper back to the distal end of the guide tube.
